(19) United States
(12) Patent Application Publication
(10) Pub. No.: US 2004/0033957 A1
(43) Pub. Date: Feb. 19, 2004

(54) FAP-activated anti-tumor prodrugs

(75) Inventors: Erik Patzelt, Purkersdorf (AU); John Edward Park, Biberach (DE); Stefan Peters, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/430,056

(22) Filed: May 6, 2003

Related U.S. Application Data
(60) Provisional application No. 60/386,163, filed on Jun. 5, 2002.

(30) Foreign Application Priority Data
May 10, 2002 (EP) 02010552.4

(51) Int. Cl. A61K 38/08; A61K 38/06; C07K 7/06; C07K 5/06

(52) U.S. Cl. 514/17; 514/18; 530/330; 530/331

(57) ABSTRACT

The present invention relates to prodrugs which are capable of being converted into prodrug intermediates of a cytotoxic or cytostatic drug, by the catalytic action of FAPα, said prodrugs exhibit

an oligomeric part comprising up to 9 amino carboxylic acid residues, the amide bond between the C-terminal amino carboxylic acid and the preceding amino acid thereof is recognized and cleaved by FAPα in the immediate environment of a target cell, and

a cytotoxic or cytostatic part, wherein the N-terminal amino function of the oligomeric part is attached to a capping group (Cg).

### RELATED APPLICATIONS

Benefit of U.S. Provisional Application Serial No. 60/386,163, filed on Jun. 5, 2002 is hereby claimed.

### TECHNICAL FIELD OF THE INVENTION

The present invention relates to the field of tumor treatment by administration of a prodrug that is converted into a drug at the site of the tumor. In particular, the invention relates to prodrugs which are converted into prodrug intermediates by the catalytic action of FAPα in the environment of a target cell, their manufacture and pharmaceutical use.

### BACKGROUND OF THE INVENTION

The human fibroblast activation protein (FAPα) is a M_{f} 95,000 cell surface molecule originally identified with monoclonal antibody (mAb) F19 (Rettig et al. (1988) Proc. Natl. Acad. Sci. USA 85, 3110-3114; Rettig et al. (1993) Cancer Res. 53, 3327-3335). The FAPα cDNA codes for a type II integral membrane protein with a large extracellular domain, trans-membrane segment, and short cytoplasmic tail (Scanlan et al. (1994) Proc. Natl. Acad. Sci. USA 91, 5657-5661; WO 97/34927). FAPα shows 48% amino acid sequence identity to the T-cell activation antigen CD26, also known as dipeptidyl peptidase IV (DPPIV; EC 3.4.14.5), a membrane-bound protein with dipeptidyl peptidase activity (Scanlan et al., loc. cit.). FAPα has enzymatic activity and is a member of the serine protease family, with serine 624 being critical for enzymatic function (WO 97/34927). Work using a membrane overlay assay revealed that FAPα dimers are able to cleave Ala-Pro-7-amino-4-trifluoromethyl coumarin, Gly-Pro-7-amino-4-trifluoromethyl coumarin, and Lys-Pro-7-amino-4-trifluoromethyl coumarin dipeptides (WO 97/34927).

FAPα is selectively expressed in reactive stromal fibroblasts of many histological types of human epithelial cancers, granulation tissue of healing wounds, and malignant cells of certain bone and soft tissue sarcomas. Normal adult tissues are generally devoid of detectable FAPα, but some fetal mesenchymal tissues transiently express the molecule. In contrast, most of the common types of epithelial cancers, including >90% of breast, non-small-cell lung, and colorectal carcinomas, contain FAPα-reactive stromal fibroblasts (Scanlan et al., loc. cit.). These FAPα⁺fibroblasts accompany newly formed tumor blood vessels, forming a distinct cellular compartment interposed between the tumor capillary endothelium and the basal aspect of malignant epithelial cell clusters (Welt et al. (1994) J. Clin. Oncol. 12(6), 1193-1203). While FAPα⁺ stromal fibroblasts are found in both primary and metastatic carcinomas, the benign and premalignant epithelial lesions tested (Welt et al., loc. cit.), such as fibroadenomas of the breast and colorectal adenomas, only rarely contain FAPα⁺ stromal cells. Based on the restricted distribution pattern of FAPα in normal tissues and its uniform expression in the supporting stroma of many malignant tumors, clinical trials with ¹³¹I-labeled mAb F19 have been initiated in patients with metastatic colon carcinomas (Welt et al., loc. cit.).

For new cancer therapies based on cytotoxic or cytostatic drugs, a major consideration is to increase the therapeutic index by improving the efficacy of cancerous tissue killing and/or reducing the toxicity for normal tissue of the cytotoxic or cytostatic agents. To increase specificity of tumor tissue killing and reduce toxicity in normal tissues, trigger mechanisms can be designed so that the toxic agents synthesized in their prodrug or inactive forms are rendered active when and where required, notably in the cancerous tissues (Panchal (1998) Biochem. Pharmacol. 55, 247-252). Triggering mechanisms may include either exogenous factors such as light or chemicals or endogenous cellular factors, such as enzymes with restricted expression in cancer tissues. Another concept, that has been further elaborated, is called ‘antibody-directed enzyme prodrug therapy’ (ADEPT) or ‘antibody-directed catalysis’ (ADC) (Huennekens (1994) Trends Biotechnol. 12, 234-239; Bagshawe (1994) Clin. Pharmacokinet. 27, 368-376; Wang et al. (1992) Cancer Res. 52, 4484-4491; Sperker et al. (1997) Clin. Pharmacokinet. 33(1), 18-31). In ADEPT, an antibody directed at a tumor-associated antigen is used to target a specific enzyme to the tumor site. The tumor-located enzyme converts a subsequently administered prodrug into an active cytotoxic agent. The antibody-enzyme conjugate (AEC) binds to a target antigen on cell membranes or to free antigen in extracellular fluid (ECF). A time interval between giving the AEC and prodrug allows for the AEC to be cleared from normal tissues so that the prodrug is not activated in the normal tissues or blood. However, some disadvantages of ADEPT are related to the properties of the AEC (Bagshawe, loc. cit.). For example, in humans, only a small fraction of the administered dose of the targeting AEC binds to tumor tissue and the remainder is distributed through body fluids from which it is cleared with significant time delays. Even very low concentrations of unbound enzyme can catalyze enough prodrug to have toxic effects because plasma and normal ECF volumes are much greater than those of tumor ECF. The AEC may also be immunogenic, thus preventing repeat administration, in many instances.

The International patent application WO 00/33888 discloses an N-L-leucyl-doxorubicin prodrug which is capable of being converted into free doxorubicin by the catalytic action of unknown enzymes, and said free doxorubicin being cytotoxic or cytostatic under physiological conditions. N-L-leucyl-doxorubicin possesses improved efficacy in mouse xenograft tumor models, [Breistol et al. (1999) Eur J Cancer 35:1143-1149] as compared to doxorubicin.

However, N-L-leucyl-doxorubicin has comparably poor tolerability, at least in humans, because roughly half of the prodrug is metabolized to free doxorubicin [Canal et al., (1992) Clin. Pharmacol. Ther. 51:249-259] directly in the circulation (whole blood or red blood cells from 107 donors could catalyze this reaction). This results in unspecific systemic exposure to doxorubicin, and therefore tremendously limits the amount of prodrug that can be administered. Myelosuppression was seen in patients and correlated with the AUC values of released doxorubicin, confirming that reduced white blood cell counts were due to generalized release of doxorubicin.

The problem underlying the present invention was to provide similar prodrugs with improved stability in whole blood, and presumably, better tolerability in humans.

### BRIEF DESCRIPTION OF THE INVENTION

The present invention relates to prodrugs which are capable of being converted into prodrug intermediate of a cytotoxic or cytostatic drug, by the catalytic action of FAPα, said prodrugs exhibit

an oligomeric part comprising up to 9 amino carboxylic acid residues, the amide bond between the C-terminal amino carboxylic acid and the following amino acid thereof is recognized and cleaved by FAPα in the immediate environment of a target cell, and

a cytotoxic or cytostatic part, wherein the N-terminal amino function of the oligomeric part is attached to a capping group (Cg).

The prodrug intermediate is cleaved within the target cell by intracellular proteases and kills the target cell or blocks its proliferation.

The prodrug is administered to the patient, carried through the blood stream in a stable form, and when in the immediate environment of a target cell, is recognized and cleaved by FAPα to release said prodrug intermediate.

In the context of this invention, a “drug” shall mean a chemical compound that may be administered to humans or animals as an aid in the treatment of disease. In particular, a drug is an active pharmacological agent.

The term “cytotoxic compound” shall mean a chemical compound which is toxic to living cells, in particular a drug that destroys or kills cells. The term “cytostatic compound” shall mean a compound that suppresses cell growth and multiplication and thus inhibits the proliferation of cells. Examples for cytotoxic or cytostatic compounds suitable for the present invention are anthracycline derivatives such as doxorubicin, analogs of methotrexate such as methothrexate, pritrexime, trimetrexate or DDMP, melphalan, analogs of cisplatin such as cisplatin, JM216, JM335, bis(platinum) or carboplatin, analogs of purines and pyrimidines such as cytarbine, gemcitabine, azacitidine, 6-thioguanine, flurdarabine or 2-deoxycoformycin, and analogs of other chemotherapeutic agents such as 9-aminocamptothecin, D,L-aminoglutethimide, trimethoprim, pyrimethamine, mitomycin C, mitoxantrone, cyclophosphanamide, 5-fluorouracil, extramustine, podophyllotoxin, bleomycin, epothilone and derivatives of epothilone as described for example in the International Patent applications WO 93/10121, WO 97/19086 or WO 98/08849 or U.S. Pat. No. 6,204,388 or taxol.

A “prodrug” shall mean a compound that, on administration, must undergo chemical conversion by metabolic processes before becoming an active pharmacological agent. In particular, a prodrug is a precursor of a drug. In the context of the present invention, the prodrug is significantly less cytotoxic or cytostatic than the drug it is converted into upon the catalytic action of FAPα. The expert knows methods of determining cytotoxicity of a compound, see e.g. example 45 herein, or Mosmann ((1983) J. Immun. Meth. 65, 55-63). Preferably, the prodrug is at least three times less cytotoxic as compared to the drug in an in vitro assay.

A “drug being cytostatic or cytotoxic under physiological conditions” shall mean a chemical compound which is cytostatic or cytotoxic in a living human or animal body, in particular a compound that kills cells or inhibits proliferation of cells within a living human or animal body.

A “prodrug having a cleavage site which is recognized by FAPα” shall mean a prodrug which can act as a substrate for the enzymatic activity of FAPα. In particular, the enzymatic activity of FAPα can catalyze cleavage of a covalent amide bond of the prodrug under physiological conditions. By cleavage of this covalent amide bond, the prodrug is converted into the drug, either directly or indirectly via a prodrug intermediate. Indirect activation would be the case if the cleavage product of the FAPα catalyzed step is not the pharmacologically active agent itself but undergoes a further reaction step, e.g. hydrolysis, to become active (i.e., is a prodrug intermediate). More preferably, the cleavage site of the prodrug is specifically recognized by FAPα but not by other proteolytic enzymes present in the human or animal body. Also preferably, the cleavage site is specifically recognised by FAPα, but not by proteolytic enzymes present in human or animal body fluids, especially plasma. In a particularly preferred embodiment, the prodrug is stable in plasma, other body fluids, or tissues, in which biologically active FAPα is not present or detectable.

The term “prodrug intermediate” as used hereinbefore and hereinbelow defines a compound obtained by the cleavage of the covalent amide bond between the C-terminal amino acid and the following amino acid by the catalytic action of FAPα of the prodrug according to the invention. Preferably the prodrug intermediate is a compound in which one amino acid is attached to a cytotoxic or cytostatic moiety.

N-L-leucyl-doxorubicin is used in an in vitro stability assay as a standard. A solution containing human whole blood (pretreated to prevent coagulation) is incubated with prodrug at 37° C. Incubations continue until 25% of the standard (N-L-leucyl-doxorubicin prodrug) is converted into free drug (free doxorubicin). Preferably, under these conditions, more than 50%, more preferably more than 70%, more preferably more than 90% of the prodrug are still present in the solution.

The cleavage site should most preferably be specific for FAPα. In a preferred embodiment, the cleavage site comprises a L-proline residue which is linked to the prodrug intermediate. The prodrug intermediate is a single amino acid linked to a cytotoxic or cytostatic drug via an amide bond. An example of a prodrug intermediate is a N-L-leucyl-doxorubicin. An example of this class is a doxorubicin-peptide conjugate. FAPα may catalyse the cleavage of a peptidic bond between the C-terminal FAP cleavage site and the prodrug intermediate, which is preferably L-leucine, coupled to the cytotoxic or cytostatic compound.

Preferred compounds show at least 10% conversion to the prodrug intermediate, under standard conditions listed below. More preferred are compounds that show at least 20% conversion to the prodrug intermediate, under standard conditions. Even more preferred are compounds that show at least 50% conversion to the prodrug intermediate, under standard conditions. In this context, standard conditions are defined as follows: Each compound is dissolved in 50 mM Hepes buffer, 150 mM NaCl, pH 7.2, at a final concentration of 5 μM and incubated with 100 ng CD8FAPα (see example 43) for 24 hours at 37° C. Release of the prodrug intermediate by CD8FAPα is determined as described in example 5 of WO 00/71571.

### DETAILED DESCRIPTION OF THE INVENTION

Preferably, the present invention relates to a compound of formula (I)

Cg-(Xaa¹)ₘ-Xaa²-Caa-Xaa³-Cyt (I)

or a pharmaceutically acceptable salt thereof,

wherein

Xaa¹ each independently represent any genetically encoded amino acid or the N-alkylated derivative thereof, at least one of which being N-terminally linked to Cg;

Xaa² represents any genetically encoded amino acid or the N-alkylated derivative thereof, which is C-terminally linked to Caa;

Xaa³ represents an amino acid selected from the group consisting of leucine, phenylalanine, isoleucine, alanine, β-alanine, glycine, tyrosine, 2-naphthylalanine and serine, or an N-alkylated derivative thereof, which is C-terminally linked to Cyt;

Caa represents an optionally substituted cyclic amino acid, which is C-terminally linked to Xaa³;

Cg represents a capping group, which blocks degradation of the attached oligopeptide moiety in body fluids;

Cyt represents the residue of a cytotoxic or cytostatic compound; and

m is 0 or an integer from 1 to 6.

An important portion of the prodrug is the capping group, which serves to protect the prodrug compound from degradation in circulating blood when it is administered to the patient and allows the prodrug to reach the environment of the target cell relatively intact. The stabilizing group protects the prodrug from degradation by proteinases and peptidases present in blood, blood serum, and normal tissue.

Particularly, since the capping group caps the N-terminus oligopeptides, and is therefore sometimes referred to as an N-cap or N-block, it serves to ward against exopeptidases to which the prodrug may otherwise be susceptible.

The compound is less toxic in vivo than the starting therapeutic agent because the prodrug is not cleaved in blood, heart, brain, bone marrow, in the mucosa and the like. This decrease in toxicity applies, in particular, to the acute effects such as marrow and mucosal toxicity, as well as possible cardiac or neurological toxicity.

Ideally, the capping group is useful in the prodrug of the invention if it serves to protect the prodrug from degradation, especially hydrolysis, when tested by storage of the prodrug compound in human blood at 37 C for 2 hours and results in less than 20%, preferably less than 2%, cleavage of the prodrug by the enzymes present in the human blood under the given assay conditions.

Accordingly, those compounds of formula I are preferred, wherein

Cg represents a capping group of formula

R¹—(CH₂)ₙ-Z-,

in which

-Z- represents —CO—, —O—CO—, —NH—CO—, —SO₂— or a single bond;

R¹ is an optionally substituted C₁-C₆-alkyl, C₃-C₈-cycloalkyl, aryl, heterocyclyl or heteroaryl group; and

n is 0, 1 or 2.

More preferred are the compounds of formula I, wherein

Cg represents a capping selected from the group consisting of succinic acid, diglycolic acid, maleic acid, polyethylene glycol, pyroglutamic acid and glutaric acid; or represents a group of formula (II)

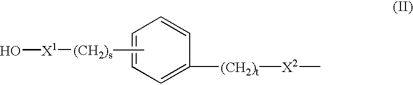

in which

X¹ represents C═O or SO₂, in particular C═O,

X² represents C═O or SO₂, in particular C═O,

s is an integer of 1 or 2, in particular 1, and

t is an integer of 1 or 2, in particular 1.

Particularly preferred are the capping groups, wherein the group HO—X¹—(CH₂)ₛ— is attached in the the meta- or para-position with respect to the group —(CH₂)ₜ—X²—. Most preferred are the capping groups selected from the formulae IIA and IIB:

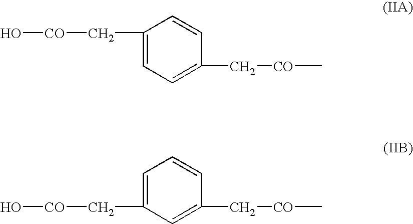

Furthermore preferred are those compounds of formula I, wherein

Caa represents a group of formula (III),

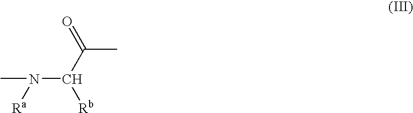

R^{a} and R^{b} together with the interjacent N—C group form an optionally substituted, optionally benzo- or cyclohexano-condensed 3- to 7-membered saturated or unsaturated heterocyclic ring, in which one or two CH₂ groups may also be replaced by NH, O or S.

Most preferably Caa is a prolin group.

Preferred are those compounds of formula I, in which

Xaa¹ and Xaa² each independently represent moieties derived from amino carboxylic acids of the formula —[NR³—(Y)_{P}—CO]— wherein Y represents CR⁴R⁵ and wherein R³, R⁴ and R⁵ each independently represent a hydrogen atom, an optionally substituted C₁-C₆-alkyl, C₃-C₈-cycloalkyl, aryl, aralkyl, heteroaryl or heteroarylalkyl group, and

p is 1, 2, 3, 4, 5; or

Xaa¹ and Xaa² each independently represent moieties derived from cyclic amino carboxylic acids of formula

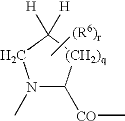

wherein

R⁶ represents C₁-C₆-alkyl, OH, or NH₂,

q is 0, 1 or 2; and

r is 0, 1 or 2.

Particular preferred are those compounds of formula I, wherein

Xaa¹ and Xaa² are amino acid moieties, which are each independently selected from glycine (Gly), and the D- or L-forms of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartatic acid (Asp), glutamic acid (Glu), asparagine (Asn), glutamine (Gin), proline (Pro), 4-hydroxy-proline (Hyp), 5-hydroxy-lysine, norleucine (Nle), 5-hydroxynorleucine (Hyn), 6-hydroxynorleucine, omithine, cyclohexylglycine (Chg), N-Methylglycin (N-MeGly), N-Methylalanin (N-MeAla), N-Methylvaline (N-MeVal), N-Methylleucine (N-MeLeu), N-Methylisoleucine (N-MeIle), N-Methylnorleucin (N-MeNle), N-Methyl-2-aminobutyric acid (N-MeAbu) and N-Methyl-2-aminopentanoic acid (N-MeNva); in particular wherein the N-terminal amino acid Xaa¹ which is directly linked to the capping group is selected from L-proline, (Pro), N-Methylglycin (N-MeGly), N-Methylalanin (N-MeAla), N-Methylvaline (N-MeVal), N-Methylleucine (N-MeLeu), N-Methylisoleucine (N-MeIle), N-Methylnorleucin (N-MeNle), N-Methyl-2-aminobutyric acid (N-MeAbu) and N-Methyl-2-aminopentanoic acid (N-MeNva).

The amino acid group Xaa¹ which is directly linked to Xaa² is preferably selected from glycine (Gly), and the D- or L-forms of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartatic acid (Asp), glutamic acid (Glu), asparagine (Asn), glutamine (Gln), proline (Pro), 4-hydroxy-proline (Hyp), 5-hydroxy-lysine, norleucine (Nle), 5-hydroxynorleucine (Hyn), 6-hydroxynorleucine, omithine, cyclohexylglycine (Chg), in particular L-alanine.

The amino acid group Xaa² is preferably selected from L-proline, glycine, L-norleucine, L-cyclohexylglycine, L-5-hydroxynorleucine, L-6-hydroxynorleucine, L-5-hydroxylysine, L-arginine, and L-lysine, in particular glycine.

The amino acid group Xaa³ which is directly linked to Cyt is preferably selected from glycine, alanine, valine, leucine, isoleucine, phenylalanine, tyrosine and serine, in particular leucine.

m is preferably 0 or an integer from 1 to 4, in particular an integer from 1 to 3, most preferably 2.

Particularly preferred are the compounds of formula IA,

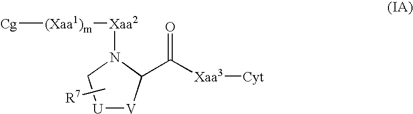

wherein Xaa¹, Xaa², Xaa³, Cg, Cyt and m are as defined hereinabove, and

U-V represents CHR⁸—CH₂, CR⁸═CH, NH—CH₂, CH₂—NH, —CR⁸—, CH₂—CHR⁸—CH₂, wherein

R⁷ and R⁸ each independently represent a hydrogen or halogen atom or a C₁-C₆-alkyl, C₁-C₆-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl group.

Highly preferred are the compounds of formula IA1,

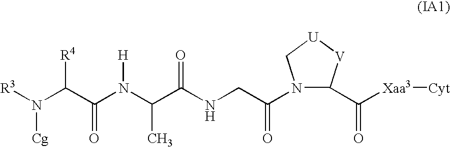

wherein R³, R⁴, Cyt, Cg, Xaa³, U and V each independently are as defined hereinbefore, or R³ and R⁴ together with the intervening N—C group form an optionally substituted, optionally benzo- or cyclohexano-condensed 3- to 7-membered saturated or unsaturated heterocyclic ring.

Most preferred are the compounds of formula I, wherein

-(Xaa¹)ₘ-Xaa²-Caa-Xaa³- represents the following amino acid sequence:

-Xaa¹-Ala-Gly-Pro-Leu-,

in which

Xaa¹ represents an N-terminal amino acid selected from the group consisting of Lys, Pro, N-MeGly, N-MeAla, N-MeVal, N-MeLeu, N-MeIle, N-MeNle, N-MeAbu and N-MeNva.

Unless indicated otherwise, the simple stereoisomers as well as mixtures or racemates of the stereoisomers are included in the invention.

“C₁-C₆-alkyl” generally represents a straight-chained or branched hydrocarbon radical having 1 to 6 carbon atoms.

The term “optionally substituted” as used hereinabove or hereinbelow with respect to a group or a moiety refers to a group or moiety which may optionally be substituted by one or several halogen atoms, hydroxyl, amino, C₁-C₆-alkyl-amino, di-C₁-C₆-alkyl-amino, C₁-C₆-alkyl-oxy, thiol, C₁-C₆-alkyl-thio, ═O, ═NH, —CHO, —COOH, —CONH₂, —NHC(═NH)NH₂, C₃-C₈-cycloalkyl, aryl, or heteroaryl substituents, which may be identical to one another or different.

The following radicals may be mentioned by way of example:

Methyl, ethyl, propyl, 1-methylethyl (isopropyl), butyl, I-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2methyl-propyl, HOCH₂—, CH₃CH(OH)—, CH₃CH(OH)CH₂CH₂—, HOCH₂CH₂CH₂CH₂—, H₂NCH₂CH₂CH₂—, H₂NCH₂CH₂CH₂CH₂—, H₂NCH₂CH(OH)CH₂CH₂—, H₂NC(═NH)NHCH₂CH₂CH₂-, HSCH₂—, CH₃SCH₂CH₂—, HOOCCH₂—, HOOCCH₂CH₂—, H₂NC(═O)CH₂—, H₂NC(═O)CH₂CH₂—, benzyl, para-hydroxy-benzyl, 4-(para-hydroxyphenoxy)-benzyl,

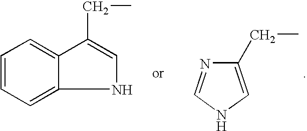

If a C₁-C₆-alkyl group is substituted, the substituents are preferably hydroxyl, amino, dimethylamino, diethylamino, thiol, methyl-thiol, methoxy, ethoxy, ═O, ═NH, —CHO, —COOH, —COOCH₃, —COOCH₂CH₃, —CONH₂, —NHC(═NH)NH₂, cyclohexyl, phenyl, benzyl, para-hydroxy-benzyl,

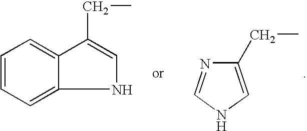

If C₁-C₆-alkyl is substituted with aryl or heteroaryl, C₁-C₆-alkyl is preferably C₁, more preferably a methylene group.

A person of ordinary skill in the chemistry of amino acids and oligopeptides will readily appreciate that certain amino acids may be replaced by other homologous, isosteric and/or isolectronic amino acids wherein the biological activity of the original amino acid or oligopeptide has been conserved upon modification. Certain unnatural and modified natural amino acids may also be utilized to replace the corresponding natural amino acid. Thus, for example, tyrosine may be replaced by 3-iodotyrosine, 2- or 3-methyltyrosine, 3-fluorotyrosine.

“C₃-C₈-Cycloalkyl” generally represents cyclic hydrocarbon radical having 3 to 8 carbon atoms which may optionally be substituted by one or several hydroxyl, amino, C₁-C₆-alkyl-amino, di-C₁-C₆-alkyl-amino, C₁-C₆-alkyl, C₁-C₆-alkyloxy, thiol, C₁-C₆-alkyl-thio, ═O, ═NH, —CHO, —COOH, —COOCH₃, —COOCH₂CH₃, —CONH₂, —NHC(═NH)NH₂, or halogen substituents, which may be identical to one another or different.

“Heterocyclic ring” as used hereinabove and hereinbelow with respect to the group formed by R^{a} and R^{b} together with the interjacent N—C group generally represents a 3 to 7-membered, preferably 4-, 5- or 6-membered non-aromatic heterocyclic ring system, containing one nitrogen atom and optionally 1 or 2 additional heteroatoms selected from the group of nitrogen, oxygen and sulfur, which may be substituted by one or several halogen atoms or C₁-C₆-alkyl, C₁-C₆-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl groups, which may be identical to one another or different, and which optionally may be benzo- or cyclohexano-condensed. Such heterocyclic rings are preferably azetidine or are derived from a fully or partially hydrogenated pyrrole, pyridine, thiazole, isoxazole, pyrazole, imidazole, indole, benzimidazole, indazole, pyridazine, pyrimidine, pyrazin group. Most preferred are azetidine, pyrrolidine, 3,4-dehydropyrrolidine, piperidine, hexahydro-1H-azepine, octahydroindole, imidazolidine, thiazolidine.

If such heterocyclic ring is substituted, the substituents are preferably methyl, ethyl, propyl, 1-methylethyl (isopropyl), butyl, 1-methylpropyl, 2-methylpropyl, 1, 1-dimethylethyl, hydroxyl, amino, dimethyl-amino, diethyl-amino, thiol, methyl-thiol, methoxy, ethoxy, —CHO, —COOH, —COOCH₃, —COOCH₂CH₃, or —CONH₂.

“Aryl” generally represents an aromatic ring system with 6 to 10, preferably 6 carbon atoms which may optionally be substituted by one or several hydroxyl, amino, C₁-C₆-alkyl-amino, di-C₁-C₆-alkyl-amino, C₁-C₆-alkyl, C₁-C₆-alkyloxy, thiol, C₁-C₆-alkyl-thio, —CHO, —COOH, —COOCH₃, —COOCH₂CH₃, —CONH₂, or halogen substituents, which may be identical to one another or different, and which optionally may be benzocondensed. Aryl substituents may be preferably derived from benzene, preferred examples being phenyl, 2-hydroxy-phenyl, 3-hydroxy-phenyl, 4-hydroxy-phenyl, 4-amino-phenyl, 2-amino-phenyl, 3-amino-phenyl.

If aryl is substituted, the substituents are preferably methyl, ethyl, propyl, 1-methylethyl (isopropyl), butyl, 1-methylpropyl, 2-methylpropyl, 1,1-dimethylethyl, hydroxyl, amino, dimethyl-amino, diethyl-amino, thiol, methyl-thiol, methoxy, ethoxy, —CHO, —COOH, —COOCH₃, —COOCH₂CH₃, or —CONH₂.

“Heteroaryl” generally represents a 5 to 10-membered aromatic heterocyclic ring system, containing 1 to 5 heteroatoms selected from the group of nitrogen, oxygen, or sulfur, which may optionally be substituted by one or several hydroxyl, amino, C₁-C₆-alkyl-amino, di-C₁-C₆-alkyl-amino, C₁-C₆-alkyl, C₁-C₆-alkyloxy, thiol, C₁-C₆-alkyl-thio, —CHO, —COOH, —COOCH₃, —COOCH₂CH₃, —CONH₂, or halogen substituents, which may be identical to one another or different, and which optionally may be benzocondensed. Heteroaryl substituents may preferably be derived from furane, pyrrole, thiophene, pyridine, thiazole, isoxazole, pyrazole, imidazole, benzofuran, thianaphthene, indole, benzimidazole, indazole, quinoline, pyridazine, pyrimidine, pyrazine, chinazoline, pyrane, purine, adenine, guanine, thymine, cytosine, uracil.

If heteroaryl is substituted, the substituents are preferably methyl, ethyl, propyl, 1-methylethyl (isopropyl), butyl, 1-methylpropyl, 2-methylpropyl, 1,1-dimethylethyl, hydroxyl, amino, dimethyl-amino, diethyl-amino, thiol, methyl-thiol, methoxy, ethoxy, —CHO, —COOH, —COOCH₃, —COOCH₂CH₃, or —CONH₂.

“Residue of a cytotoxic or cytostatic compound” means that the compound H₂N-Cyt′, which is released upon cleavage of the amide bond shown in formula (I), is either cytotoxic or cytostatic itself, or may be converted into a cytotoxic or cytostatic compound in a subsequent step.

In the latter case, -Cyt′ may be a residue of formula -L-Cyt″, wherein L is a linker residue derived from a bifunctional molecule, for instance a diamine H₂N-L′-NH₂, an amino alcohol H₂N-L′-OH, for example p-amino-benzyl alcohol (PABOH), an amino carbonate, for example

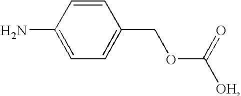

or an unnatural amino carboxylic acid. If -Cyt′ is of formula -L-Cyt″, the compound H₂N-L′-Cyt″ is generated by the enzymatic cleavage of the amide bond shown in formula (I). The compound H₂N-L′-Cyt″ may be cytotoxic or cytostatic itself or the linker residue cleaved off from Cyt″ in a subsequent step releasing the cytotoxic or cytostatic agent. For example, the compound H₂N-L′-Cyt″ may be hydrolysed under physiological conditions into a compound H₂N-L′-OH and the cytotoxic or cytostatic compound H-Cyt″, which is the active therapeutic agent (In the following, only the term Cyt′ is used for both Cyt′ and Cyt″, and only the term L is used for both L and L′, for simplicity).

The pharmaceutically acceptable salts of the compounds of the present invention include the conventional non-toxic salts formed from non-toxic inorganic or organic acids. For example, such conventional non-toxic salts include those from inorganic acids such as hydrochloric acid, hydrobromic, sulfuric, sulfamic, phosphoric, nitric and the like; and the salts prepared from organic acids such as acetic, propionic, succinic, glycolic, stearic, lactic, malic, tartaric, citric, ascorbic, maleic, phenylacetic, glutamic, benzoic, salicylic, sulfanilic, oxalictrifluoroacetic and the like.

H₂N-Cyt′ is preferably an anthracycline derivative of formula IV,

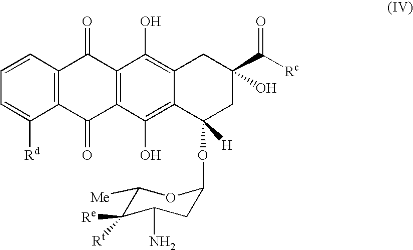

wherein

R^{c} represents C₁-C₆ alkyl, C₁-C₆ hydroxyalkyl or C₁-C₆ alkanoyloxy C₁-C₆ alkyl, in particular methyl, hydroxymethyl, diethoxyacetoxymethyl or butyryloxymethyl;

R^{d} represents hydrogen, hydroxy or C₁-C₆ alkoxy, in particular methoxy;

one of R^{e} and R^{f} represents a hydrogen atom; and the other represents a hydrogen atom or a hydroxy or tetrahydropyran-2-yloxy (OTHP) group.

Paricularly preferred are the following compounds of formula IV:

| R^{c} | R^{d} | R^{e} | R^{f} | Cyt |
|---|---|---|---|---|
| CH₂OH | OCH₃ | H | OH | doxorubicin |
| CH₃ | OCH₃ | H | OH | daunorubicin |
| CH₂OH | OCH₃ | OH | H | epirubicin |
| CH₃ | H | H | OH | idarubicin |
| CH₂OH | OCH₃ | H | OTHP | THP |
| CH₂OH | OCH₃ | H | H | esorubicin |
| CH₂OCOCH(OC₂H₅)₂ | OCH₃ | H | OH | detorubicin |
| CH₂OH | H | H | OH | carminorubicin |
| CH₂OCOC₄H₉ | OCH₃ | H | OH | |

Most preferred is doxorubicin (Dox). Other cytotoxic or cytostatic residues Cyt′ may be derived for example from methotrexate, trimetrexate, pyritrexim, 5,10-dideazatetrahydrofolatepyrimetamine, trimethoprim, 10-propargyl-5,8-dideazafolate2,4-diamino-5 (3′,4′-dichloropheyl)-6-methylpyrimidine, aminoglutethimide, goreserelin, melphalan, chlorambucil, analogs of other chemotherapeutic agents such as 9-aminocamptothecin (for examples see e.g. Burris HA, r. d. and S. M. Fields (1994). “Topoisomerase I inhibitors. An overview of the camptothecin analogs. [Review].” Hematol. Oncol. Clin. North Am. 8(2): 333-355; Iyer, L. and M. J. Ratain (1998). “Clinical pharmacology of camptothecins. [Review] [137 refs].” Cancer Chemother. Pharmacol. 42 Suppl: S31-S43.) and epothilone and the derivatives thereof.

In formula (I), Cyt′ may also be a biological effector molecule which either directly or indirectly effects destruction of tumor cells, like for example TNFα.

Preferred anthracycline prodrugs are the compounds of formulae (IA1-1) to (IA1-3), wherein Cg is as defined hereinbefore:

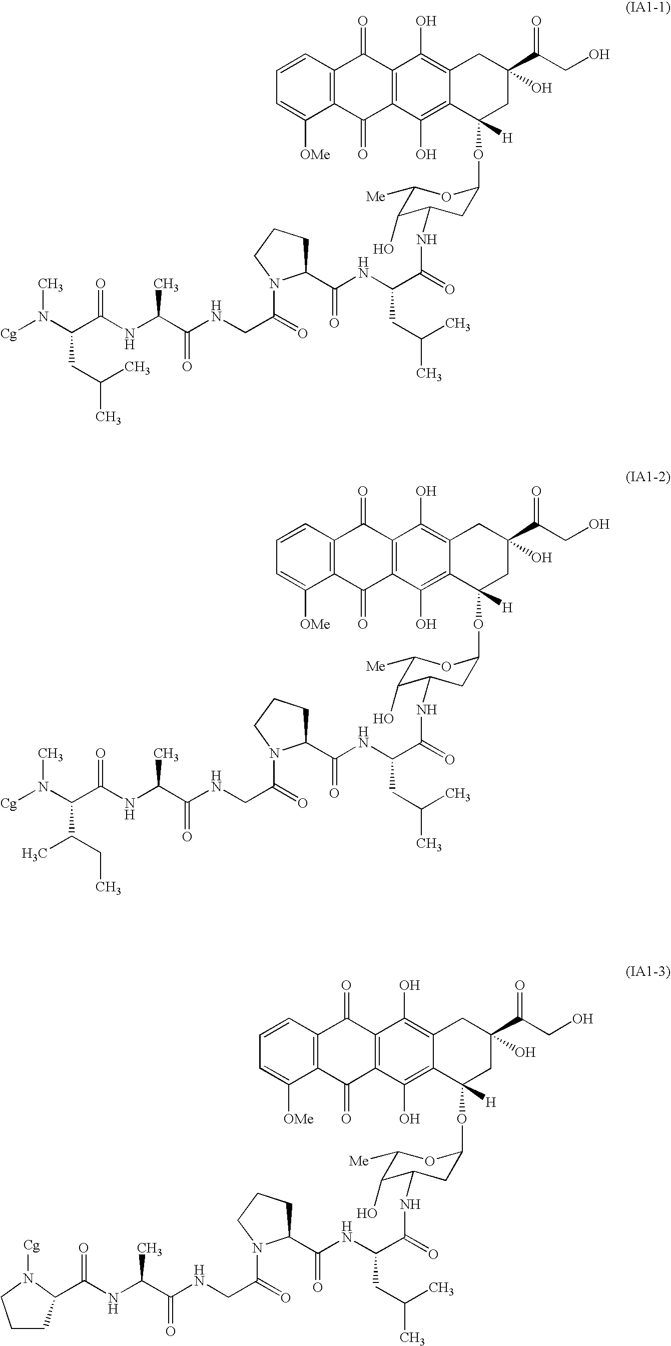

Most preferred compounds of the invention are doxorubicin derivatives of formulae (IA1-4) to (IA1-8):

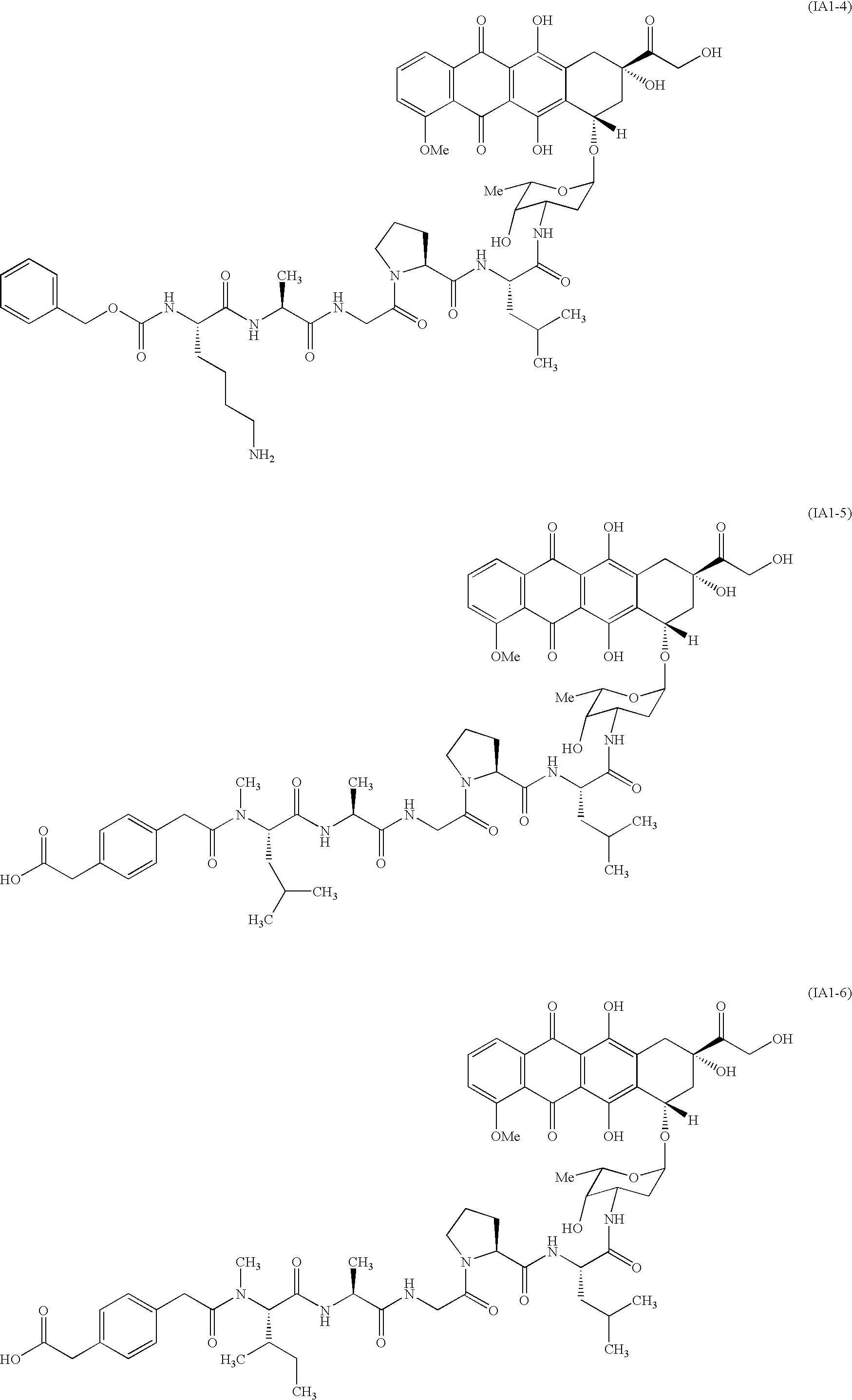

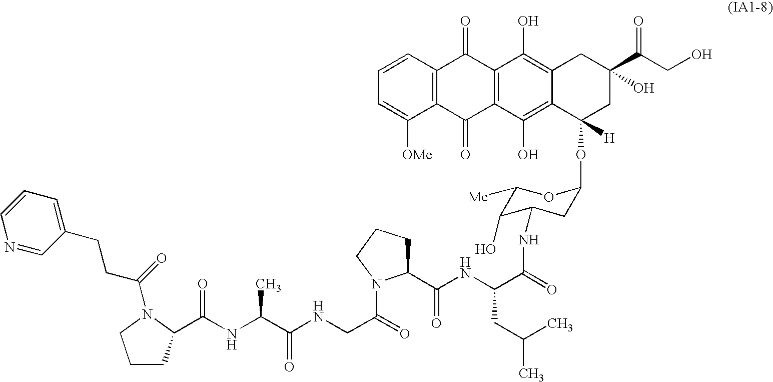

If the oligomeric part Cg-(Xaa¹)ₘ-Xaa²-Caa-Xaa³- of formula (I) contains two or more sulfur atoms, the compound of the invention may contain one or more disulfide bonds.

One class of cytotoxic or cytostatic compounds which may be used for the present invention has a primary amino function which is available for formation of an amidic bond as shown in formula (I), like doxorubicin. In this case, a linker molecule L is not necessary. If a cytostatic or cytotoxic compound does not have such an amino function, such a function may be created in such a compound by way of chemical modification, e.g. by introducing or converting a functional group or attaching a linker molecule to the compound. A linker molecule may also be inserted between the oligomeric part (i.e. the part comprising the amino carboxylic residues) and the cytostatic or cytotoxic part of the compound of the invention to ensure or optimize cleavage of the amide bond between the oligomeric part and the cytotoxic or cytostatic part. If a linker molecule is present, i.e. in compounds containing the structure L-Cyt′, the bond between L and Cyt′ is preferably an amidic or ester bond. In a preferred embodiment, such a linker molecule is hydrolyzed off the cytostatic or cytotoxic compound under physiological conditions after the enzymatic cleavage and thus the free cytostatic or cytotoxic compound is generated. In any case, the compound of the invention must have the property of being cleavable upon the catalytic action of FAPα and, as a direct or indirect consequence of this cleavage, releasing under physiological conditions a cytostatic or cytotoxic compound.

In a further aspect, the present invention relates to prodrug, which exhibits

an oligomeric part comprising up to 9 amino carboxylic acid residues, and

a cytotoxic or cytostatic part (Cyt), wherein the N-terminal amino function of the oligomeric part is attached to a capping group (Cg); wherein

the amide bond between the C-terminal amino carboxylic acid (Xaa³) and the following amino acid, in particular Caa, thereof is recognized and cleaved by FAPα in the immediate environment of a target cell to obtain a prodrug intermediate of formula (IV),

H-Xaa³-Cyt (IV)

wherein Xaa³ and Cyt have the meaning given.

Preferably, the oligomeric part comprises two, three, four, five, six, seven, eight or nine amino carboxylic acid residues, more preferably two, three, four or five amino carboxylic residues, in particular five amino acid residues.

The compounds of the invention may be synthesized by processes known in the art (E. Wünsch, Synthese von Peptiden, in “Methoden der organischen Chemie”, Houben-Weyl (Eds. E. Müller, O. Bayer), Vol. XV, Part 1 and 2, Georg Thieme Verlag, Stuttgart, 1974).

The peptide, or oligopeptide, sequences in the prodrug conjugates of this invention may be synthesized by the solid phase peptide synthesis (using either t-butyloxycarbonyl (Boc) or 9-fluorenylmethyloxycarbonyl (Fmoc) chemistry) methods or by solution phase synthesis. The general Boc and Fmoc methods are widely used and are described in the following references: Merrifield, J. A. Chem. Soc., 88: 2149 (1963); Bodanszky and Bodanszky, The Practice of Peptide Synthesis, Springer-VerlaQ, Berlin, 7-161 (1994); Stewart, Solid Phase Peptide Synthesis, Pierce Chemical, Rockford, (1984).

Using the preferred solid phase synthesis method, either automated or manual, a peptide of desired length and sequence is synthesized through the stepwise addition of amino acids to a growing chain which is linked to a solid resin. Examples of useful Fmoc compatible resins, but not limited to, are Wang resin, HMPA-PEGA resin, Rink acid resin, or a hydroxyethyl-photolinker resin. The C-terminus of the peptide chain is covalently linked to a polymeric resin and protected a-amino amino acids were added in a stepwise manner with a coupling reagent. A preferred a-amino protecting group is the Fmoc group, which is stable to coupling conditions and can readily be removed under mild alkyline conditions. The reaction solvents are preferably but not limited to dimethylformamide (DMF), N-methylpyrrolidine (NMP), dichloromethane (DCM), methanol (MeOH), and ethanol (EtOH). Examples of coupling agents are: dicyclohexylcarbodiimide (DCC), diisoprpoylcarbodiimide (DIC), O-(7-Azabenzotriazol-1-yl)-1,1,3,3-tetramethyluroniumhexafluorophosphate (HATU), 2-(1H-benzotriazol-1-yl-)-1,1,3,3-tetramethyluroniumhexafluorophosphate (HBTU). Cleavage of the N-terminal protecting group is accomplished in 10-100% piperidine in DMF at 0-40 C, with ambient temperature being preferred. At the end of synthesis the final Fmoc protecting group is removed using the above N-terminal cleavage procedure. The remaining peptide on resin is cleaved from the resin along with any acid sensitive side chain protecting groups by treating the resin under acidic conditions. For example an acidic cleavage condition is a mixture of trifluroacetic acid (TFA) in dichloromethane. If the hydroxyethylphotolinker resin is used, the appropriate wavelength for inducing cleavage is X 365 nm ultraviolet light.

The preparation of N-terminus derivatized peptides is conveniently accomplished on solid phase. When the peptide synthesis is complete and the terminal Fmoc is removed while the peptide is still on the solid support. The capping group (Cg) of choice is coupled next using standard peptide coupling conditions onto the N terminus of the peptide. On completion of the Cg coupling the peptide is cleaved from the resin using the procedure described above.

For the solid phase method using Boc chemistry, either the Merrifield resin or 4-hydroxy-methylphenylacetamidomethyl (PAM) resin is useful. The amino acids are coupled to the growing chain on solid phase by successive additions of coupling agent activated Boc-protected amino acids.

Examples of coupling agents are: DCC, DIC, HATU, HBTU. The reaction solvents may be DMF, DCM, MeOH, and NMP. Cleavage of the Boc protecting group is accomplished in 10-100% TFA in DCM at 0-40° C., with ambient temperature being preferred. On completion of the peptide chain assembly the N-terminus protecting group (usually Boc) is removed as described above. The peptide is removed from the resin using liquid HF or trifluoromethane sulfonic acid in dichloromethane.

Alternatively, the peptide intermediate may be made via a solution phase synthesis, utilizing either Boc or Fmoc chemistry. The peptide can be built up by the stepwise assembly in analogy to the solid phase method (in the N-terminal direction or in the C-terminal direction) or through the coupling of two suitably protected dipeptides or a tripeptide with a single amino acid.

On completion of the oligopeptide assembly the N-terminus deprotected and the C-terminus protected peptide is ready to accept the desired capping group

When constructing the capping group by solution phase synthesis, the capping group needs to be synthesized by a slightly modified procedure. First the C-terminus of the Fmoc oligopeptide needs to be protected with an acid labile or hydrogenation sensitive protecting group compatible with the selective deprotection of the C-terminus over the capping group. Then the Fmoc protecting group needs to be removed from the oligopeptide to reveal the N-terminus. With the N-terminus deprotected, and the C-terminus protected the oligopeptide is reacted with the activated hemi-ester of the desired Cg. The Cg can be activated using methods for activating amino acids such as DCC or HATU in base and an appropriate solvent.

Any combination of the above method can be considered, such as “fragment condensation” of di-, or tripeptides. The reaction conditions are well known in the art and detailed in the citations given. The advantage of the above described methods is the facile purification of the product produced by solution phase synthesis.

For example, the compounds of formula I, wherein Cg is a capping group of formula II, can be synthesized by condensation of the terminal amino function of the oligomeric part of a compound of formula VI with a compound of formula V,

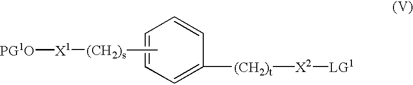

wherein X¹, X², s and t have the meaning given hereinabove, and

LG¹ is OH or an activation leaving group, and PG¹ is H or a suitable protection group

according to the following reaction scheme:

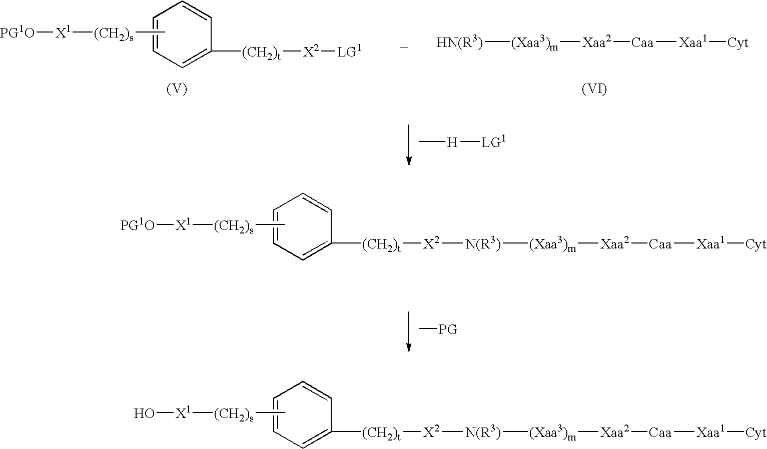

wherein Cyt, Xaa¹, Xaa², Xaa³, Caa, R³ and m have the meaning given hereinabove.

To achieve such an amide formation, it may be necessary to activate the carbonyl group of the carboxylic acid for a nucleophilic attack of an amine, i.e. LG¹ to be an activation group or leaving group which is suitable to be substituted by an amino group. This activation can be done by conversion of the carboxylic acid into an acid chloride or acid fluoride or by conversion of the carboxylic acid into an activated ester, for instance a N-hydroxysuccinimidyl ester or a pentafluorophenyl ester. Another method of activation is the transformation into a symmetrical or unsymmetrical anhydride. Alternatively, the formation of the amide bonds can be achieved by the use of in situ coupling reagents like benzotriazole-1-yl-oxy-tris-pyrrolidino-phosphonium hexafluorophosphate (PyBOP) (E. Frerot et al., Tetrahedron, 1991, 47, 259-70), 1,1′-carbonyldimidazole (CDI) (K. Akaji et al., THL, 35, 1994, 3315-18), 2-(1H-benzotriazole-1-yl)-1,1,3,3-tetramethyluronium tetrafluoroborate (TBTU) (R. Knorr et al., THL, 30, 1989, 1927-30), 1-(mesitylene-2-sulfonyl)-3-nitro-1H-1,2,4-triazole (MSNT) (B. Blankenmeyer-Menge et al., THL, 31, 1990, 1701-04).

The protection group PG¹ is removed at the end of the synthesis.

The compounds of formula VI can be prepared as described in WO 00/71571, the complete disclosure of which is hereby incorporated by reference.

The compounds of the invention are intended for medical use. In particular, these compounds are useful for the treatment of tumors which are associated with stromal fibroblasts that express FAPα and which are generally not optimally treated with available cytotoxic and/or cytostatic agents. Tumors with this property are, for example, epithelial cancers, such as lung, breast, and colon carcinomas. Tumors, such as bone and soft tissue sarcomas which express FAPα, may also be treated with these compounds.

Consequently, another aspect of the present invention are pharmaceutical compositions comprising a compound of the present invention and optionally one or more suitable and pharmaceutically acceptable excipients, as exemplified in: Remington: the science and practice of pharmacy. 19th ed. Easton. Mack Publ., 1995. The pharmaceutical compositions may be formulated as solids or solutions. Solid formulations may be for preparation of a solution before injection.

The pharmaceutical composition may, for example, be administered to the patient parenterally, especially intravenously, intramuscularly, or intraperitoneally. Preferably, the pharmaceutical compositions of the invention are solutions for injection.

Pharmaceutical compositions of the invention for parenteral administration comprise sterile, aqueous or nonaqueous solutions, suspensions, or emulsions. As a pharmaceutically acceptable solvent or vehicle, propylene glycol, polyethylene glycol, injectable organic esters, for example ethyl oleate, or cyclodextrins may be employed. These compositions can also comprise wetting, emulsifying and/or dispersing agents.

The sterilization may be carried out in several ways, for example using a bacteriological filter, by incorporating sterilizing agents in the composition or by irradiation. They may also be prepared in the form of sterile solid compositions which may be dissolved at the time of use in sterile water or any other sterile injectable medium.

The pharmaceutical composition may also comprise adjuvants which are well known in the art (e.g., vitamin C, antioxidant agents, etc.) and capable of being used in combination with the compound of the invention in order to improve and prolong the treatment of the medical condition for which they are administered.

Doses for administration to a patient of the compounds according to the invention are generally at least the usual doses of the therapeutic agents known in the field and will be adjusted according to factors like body weight and health status of the patient, nature of the underlying disease, therapeutic window of the compound to be applied, solubility, and the like, as described for example in Bruce A. Chabner and Jerry M. Collins, Cancer Chemotherapy, Lippincott Ed., ISBN 0-397-50900-6 (1990) or they may be adjusted, within the judgment of the treating physician, to accommodate the superior effectiveness of the prodrug formulations or the particular circumstances of the patient being treated. The doses administered hence vary in accordance with the therapeutic agent used for the preparation of the compound according to the invention.

For doxorubicin conjugates, for example, the dose will preferably be in the range from 10 mg/m² to 2000 mg/m², but also higher or lower doses may be appropriate.

Accordingly, a further aspect of the present invention is the use of a compound of the invention in the preparation of a pharmaceutical composition for the treatment of cancer. Furthermore, an aspect of the invention is a method of treatment of cancer, comprising administering an effective amount of a pharmaceutical composition of the invention to a patient. Indications include the treatment of cancer, specifically:

1) The treatment of epithelial carcinomas including breast, lung, colorectal, head and neck, pancreatic, ovarian, bladder, gastric, skin, endometrial, ovarian, testicular, esophageal, prostatic and renal origin;

2) Bone and soft-tissue sarcomas: Osteosarcoma, chondrosarcoma, fibrosarcoma, malignant fibrous histiocytoma (MFH), leiomyosarcoma;

3) Hematopoietic malignancies: Hodgkin's and non-Hodgkin's lymphomas;

4) Neuroectodermal tumors: Peripheral nerve tumors, astrocytomas, melanomas;

5) Mesotheliomas.

Also included are the treatment of chronic inflammatory conditions such as rheumatoid arthritis, osteoarthritis, liver cirrhosis, lung fibrosis, arteriosclerosis, and abnormal wound healing.

A further aspect of the invention is a method of treatment of cancer, wherein a prodrug is administered to a patient wherein said prodrug is capable of being converted into a cytotoxic or cytostatic drug by an enzymatic activity, said enzymatic activity being the expression product of cells associated with tumor tissue. Preferably, said enzymatic activity is the proteolytic activity of FAPα.

One method of administration of the compounds is intravenous infusion. Other possible routes of administration include intraperitoneal (either as a bolus or infusion), intramuscular or intratumoral injection. Where appropriate, direct application may also be possible (for example, lung fibrosis).

All publications and patent applications mentioned in this specification are herein incorporated by reference to the same extent as if each individual publication or patent application was specifically and individually indicated to be incorporated by reference.

One skilled in the art will appreciate that although specific reagents and reaction conditions are outlined in the following examples, modifications can be made which are meant to be encompassed by the scope of the invention. The following examples, therefore, are intended to further illustrate the invention and are not limiting.

### EXAMPLE 1

### Synthesis of (4-Carboxymethyl-phenyl)-acetic Acid N-hydroxysuccinimidyl Ester

1,4-phenylendiacetic acid (4.8 g, 25 mmol) was dissolved in acetonitrile (100 ml) and N,N-dimethylformamide (100 ml) The solution was cooled to 0° C. and N-hydroxysuccinimide (2.9 g, 25 mmol) and diisopropylethylamine (8.6 ml, 50 mmol) were added. The solution was stirred for 30 min at 0° C. Then a solution of dicyclohexyl carbodiimide (5.2 g, 25 mmol) dissolved in acetonitrile (50 ml) was added dropwise over 1 h. The ice bath was removed an the suspension was stirred over night. The suspension was filtered and the solvent of the filtrate was concentrated under reduced pressure. A residual amount of dicyclohexyl urea was precipitated by addition of diethyl ether and removed by filtration. The filtrate was purified by reversed phase HPLC applying acetonitrile/water gradient. The product fraction was lyophilized to give the product as white crystals (1.1 g, 16%). The product gave satisfactory analytical data.

[2-(4-Carboxymethyl-phenyl)-acetyl]-N-MeLeu-Ala-Gly-Pro-Leu-Dox

All steps were performed under nitrogen atmosphere.

Aloc-N-MeLeu-Ala-Gly-Pro-Leu-Dox:

Aloc-N-MeLeu-Ala-Gly-Pro-Leu-OH (0.45 mmol) and Doxorubicin hydrochloride (0.49 mmol) were dissolved in N,N-dimethylformamide (5 ml). Diisopropylethylamine (153 μl 0.90 mmol) was added and the solution was cooled to 0° C. To the stirred mixture a solution of O-(7-azabenzotriazol-1-yl)-1,1,3,3-tetramethyluronium hexfluorophosphate (HATU) (0.582 mmol) in N,N-dimethylformamide (10 ml) was added dropwise over a period of 30 min. The solution was stirred for 1 h at 0° C. After addition of diisopropylethylamine (25 μl, 0.14 mmol) the solvent was removed in vacuo at 30° C. The crude product was dissolved in methanol (1 ml) and water (6 ml) and purified by reversed phase HPLC applying acetonitrile/water gradient. The product fraction was lyophilized and gave red cristalls of Aloc-Pro-Ala-Gly-Pro-Leu-Dox (81%). The product gave satisfactory analytical data.

H-N-MeLeu-Ala-Gly-Pro-Leu-Dox:

Aloc-N-MeLeu-Ala-Gly-Pro-Leu-Dox (0.36 mmol) was dissolved dichloromethane (20 ml) and diethylamine (185 μl, 1.81 mmol) and Pd(Ph₃P)₄ (0.02 mmol) were added. The solution was stirred at room temperature for 1 h. The solvent was removed under reduced pressure and the crude oil was dissolved water (3 ml) and acetonitrile (1 ml). The product was purified by reversed phase HPLC applying acetonitrile/water gradient and the product fraction was lyophilized to give red crystals of H-Pro-Ala-Gly-Pro-Leu-Dox (95%). The product gave satisfactory analytical data.

[2-(4-Carboxymethyl-phenyl)-acetyl]-N-MeLeu-Ala-Gly-Pro-Leu-Dox:

H-N-MeLeu-Ala-Gly-Pro-Leu-Dox (0.12 mmol) was dissolved in in N,N-dimethylformamide (5 ml). (4-Carboxymethyl-phenyl)-acetic acid N-hydroxysuccinimidyl ester (0.127 mmol) and diisopropylethylamine (22 μl 0.127 mmol) were added. The solution was stirred for 48 b at room temperature. The solvent was removed under reduced pressure at 30° C. The crude product was purified by reversed phase HPLC applying acetonitrile/water gradient and the product fraction was concentrated by lyophilization to yield the product (77 mg, 64%). The product gave satisfactory analytical data.

The following prodrugs are obtained analogously:

[2-(4-Carboxymethyl-phenyl)-acetyl]-N-Pro-Ala-Gly-Pro-Leu-Dox

[2-(4-Carboxymethyl-phenyl)-acetyl]-N-Lys-Ala-Gly-Pro-Leu-Dox

[2-(4-Carboxymethyl-phenyl)-acetyl]-N-Lys-Ala-Gly-Pro-Leu-Dox

[2-(4-Carboxymethyl-phenyl)-acetyl]-N-MeIle-Ala-Gly-Pro-Leu-Dox

[2-(4-Carboxymethyl-phenyl)-acetyl]-N-MeVal-Ala-Gly-Pro-Leu-Dox

[2-(4-Carboxymethyl-phenyl)-acetyl]-Aze-Ala-Gly-Pro-Leu-Dox

[2-(3-Carboxymethyl-phenyl)-acetyl]-N-MeIle-Ala-Gly-Pro-Leu-Dox

[2-(3-Carboxymethyl-phenyl)-acetyl]-N-MeLeu-Ala-Gly-Pro-Leu-Dox

[2-(3-Carboxymethyl-phenyl)-acetyl]-N-MeVal-Ala-Gly-Pro-Leu-Dox

Each compound is dissolved in 50 mM Hepes buffer, 150 mM NaCl, pH 7.2, at a final concentration of 5 μM and incubated with 100 ng CD8FAPα for 24 hours at 37° C. Release of the prodrug intermediate Leu-Dox by CD8FAPα is determined as described in example 5 of WO 00/71571.

1. A compound of the formula (I)
Cg-(Xaa¹)ₘ-Xaa²-Caa-Xaa³-Cyt (I)
or a pharmaceutically acceptable salt thereof, wherein:
Xaa¹ each independently represent any genetically encoded amino acid or the N-alkylated derivative thereof, at least one of which being N-terminally linked to Cg;
Xaa² represents any genetically encoded amino acid or the N-alkylated derivative thereof, which is C-terminally linked to Caa;
Xaa³ represents an amino acid selected from the group consisting of leucine, phenylalanine, isoleucine, alanine, β-alanine, glycine, tyrosine, 2-naphthylalanine and serine, or an N-alkylated derivative thereof, which is C-terminally linked to Cyt;
Caa represents an optionally substituted cyclic amino acid, which is C-terminally linked to Xaa³;
Cg represents a capping group, which blocks degradation of the attached oligopeptide moiety in body fluids;
Cyt represents the residue of a cytotoxic or cytostatic compound; and
m is 0 or an integer from 1 to 6:

2. A compound of the formula (I) according to claim 1, wherein:
Cg represents a capping group of formula
R¹—(CH₂)ₙ-Z-,
in which:
-Z- represents —CO—, —O—CO—, —NH—CO—, —SO₂— or a single bond;
R¹ is an optionally substituted C₁-C₆-alkyl, C₃-C₈-cycloalkyl, aryl, heterocyclic or heteroaryl group; and
n is 0, 1 or 2.

3. A compound of the formula (I) according to claim 1, wherein:
Cg represents a capping group selected from the group consisting of succinic acid, diglycolic acid, maleic acid, polyethylene glycol, pyroglutamic acid and glutaric acid; or Cg represents a capping group of the formula (II) 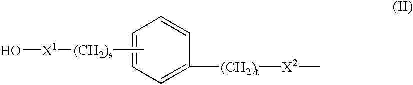
in which:
X¹ represents C═O or SO₂,
X² represents C═O, SO₂, NH—C═O or a single bond,
s is an integer of 1 or 2, and
t is 0 or an integer of 1, 2 or 3.

4. A compound of formula (I) according to claim 1 wherein
Caa represents a group of formula (III), 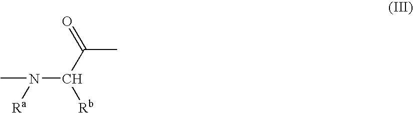
in which R^{a} and R^{b} together with the intervening N—C group form an optionally substituted, optionally benzo- or cyclohexano-condensed 3- to 7-membered saturated or unsaturated heterocyclic ring, in which one or two CH₂ groups may also be replaced by NH, O or S.

5. A compound of formula (I) according to claim 3, wherein
Cg represents a capping group of the formula (II), in which:
X¹ represents C═O or SO₂,
X² represents C═O or SO₂,
s is an integer of 1 or 2, and
t is an integer of 1 or 2.

6. A compound of formula (I) according to claim 5, wherein X¹ and X² represent C═O and
s and t are 1.

7. A compound of formula (I) according to any of the preceding claims, wherein:
Xaa¹ and Xaa² each independently represent moieties derived from amino carboxylic acids of the formula
—[NR³—(Y)ₚ—CO]—
wherein:
Y represents CR⁴R⁵,
R³, R⁴ and R⁵ each independently represent a hydrogen atom, an optionally substituted C₁-C₆-alkyl, C₃-C₈-cycloalkyl, aryl, aralkyl, heteroaryl or heteroarylalkyl group, and
p is 1, 2, 3, 4, 5; or
Xaa¹ and Xaa² each independently represent moieties derived from cyclic amino carboxylic acids of formula 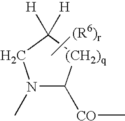
wherein:
R⁶ represents C₁-C₆-alkyl, OH, or NH₂,
q is 0, 1 or 2; and
r is 0, 1 or 2.

8. A compound of formula (IA), 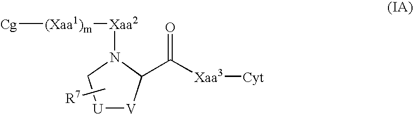
wherein:
Xaa¹ each independently represent any genetically encoded amino acid or the N-alkylated derivative thereof, at least one of which being N-terminally linked to Cg;
Xaa² represents any genetically encoded amino acid or the N-alkylated derivative thereof, which is C-terminally linked to Caa;
Xaa³ represents an amino acid selected from the group consisting of leucine, phenylalanine, isoleucine, alanine, β-alanine, glycine, tyrosine, 2-naphthylalanine and serine, or an N-alkylated derivative thereof, which is C-terminally linked to Cyt;
Cg represents a capping group, which blocks degradation of the attached oligopeptide moiety in body fluids;
Cyt represents the residue of a cytotoxic or cytostatic compound;
m is 0 or an integer from 1 to 6;, and
R⁷ represents a hydrogen or halogen atom or a C₁-C₆-alkyl, C1-C6-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl group; and
U-V represents CHR⁸—CH₂, CR⁸═CH, NH—CH₂, CH₂—NH, —CR⁸— or CH₂—CHR⁸—CH₂, wherein:
R⁸ represent a hydrogen or halogen atom or a C₁-C₆-alkyl, C₁-C₆-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl group.

9. A compound of formula (IA1), 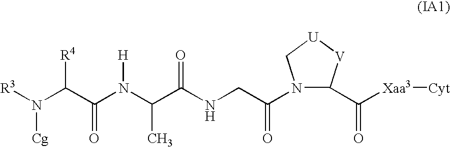
wherein:
R³ and R⁴ each independently represent a hydrogen atom, an optionally substituted C1-C6-alkyl, C3-C8-cycloalkyl, aryl, aralkyl, heteroaryl or heteroarylalkyl group; or
R³ and R⁴ together with the interjacent N—C group form an optionally substituted, optionally benzo- or cyclohexano-condensed 3- to 7-membered saturated or unsaturated heterocyclic ring;
Cg represents a capping group, which blocks degradation of the attached oligopeptide moiety in body fluids;
Cyt represents the residue of a cytotoxic or cytostatic compound; and
U-V represents CHR⁸—CH₂, CR⁸═CH, NH—CH₂, CH₂—NH, —CR⁸— or CH₂—CHR⁸—CH₂, wherein:
R⁸ represents a hydrogen or halogen atom or a C₁-C₆-alkyl, C₁-C₆-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl group.

10. A compound of the formula (I) according to claim 1, wherein Xaa¹ and Xaa² are amino acid moieties, which are each independently selected from glycine (Gly), and the D- or L-forms of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartatic acid (Asp), glutamic acid (Glu), asparagine (Asn), glutamine (Gln), proline (Pro), 4-hydroxy-proline (Hyp), 5-hydroxy-lysine, norleucine (Nle), 5-hydroxynorleucine (Hyn), 6-hydroxynorleucine, ornithine, cyclohexylglycine (Chg), N-Methylglycin (N-MeGly), N-Methylalanin (N-MeAla), N-Methylvaline (N-MeVal), N-Methylleucine (N-MeLeu), N-Methylisoleucine (N-MeIle), N-Methylnorleucin (N-MeNle), N-Methyl-2-aminobutyric acid (N-MeAbu) and N-Methyl-2-aminopentanoic acid (N-MeNva).

11. A compound of the formula (IA) according to claim 8, wherein Xaa¹ and Xaa² are amino acid moieties, which are each independently selected from glycine (Gly), and the D- or L-forms of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartatic acid (Asp), glutamic acid (Glu), asparagine (Asn), glutamine (Gin), proline (Pro), 4-hydroxy-proline (Hyp), 5-hydroxy-lysine, norleucine (Nle), 5-hydroxynorleucine (Hyn), 6-hydroxynorleucine, ornithine, cyclohexylglycine (Chg), N-Methylglycin (N-MeGly), N-Methylalanin (N-MeAla), N-Methylvaline (N-MeVal), N-Methylleucine (N-MeLeu), N-Methylisoleucine (N-MeIle), N-Methylnorleucin (N-MeNle), N-Methyl-2-aminobutyric acid (N-MeAbu) and N-Methyl-2-aminopentanoic acid (N-MeNva).

12. A compound of formula (I) according to claim 1, wherein the group Xaa¹ which is directly linked to the capping group is selected from L-proline, (Pro), N-Methylglycin (N-MeGly), N-Methylalanin (N-MeAla), N-Methylvaline (N-MeVal), N-Methylleucine (N-MeLeu), N-Methylisoleucine (N-MeIle), N-Methylnorleucin (N-MeNle), N-Methyl-2-aminobutyric acid (N-MeAbu) and N-Methyl-2-aminopentanoic acid (N-MeNva).

13. A compound of formula (IA) according to claim 8, wherein the group Xaa¹ which is directly linked to the capping group is selected from L-proline, (Pro), N-Methylglycin (N-MeGly), N-Methylalanin (N-MeAla), N-Methylvaline (N-MeVal), N-Methylleucine (N-MeLeu), N-Methylisoleucine (N-MeIle), N-Methylnorleucin (N-MeNle), N-Methyl-2-aminobutyric acid (N-MeAbu) and N-Methyl-2-aminopentanoic acid (N-MeNva).

14. A compound of the formula (I) according to claim 1, wherein the amino acid moieties exist in the (L)-configuration.

15. A compound of formula (IA) according to claim 8, wherein the amino acid moieties exist in the (L)-configuration.

16. A compound of the formula (IA1) according to claim 9, wherein the amino acid moieties exist in the (L)-configuration.

17. A compound of the formula (I) according to claim 1, wherein Cyt is an anthracycline group.

18. A compound of the formula (IA) according to claim 8, wherein Cyt is an anthracycline group.

19. A compound of the formula (IA1) according to claim 9, wherein Cyt is an anthracycline group.

20. A compound of the formula (I) according to claim 1, wherein Xaa³ is leucine.

21. A compound of formula (IA) according to claim 8, wherein Xaa³ is leucine.

22. A compound of formula (IA1) according to claim 9, wherein Xaa³ is leucine.

23. A compound of the formula 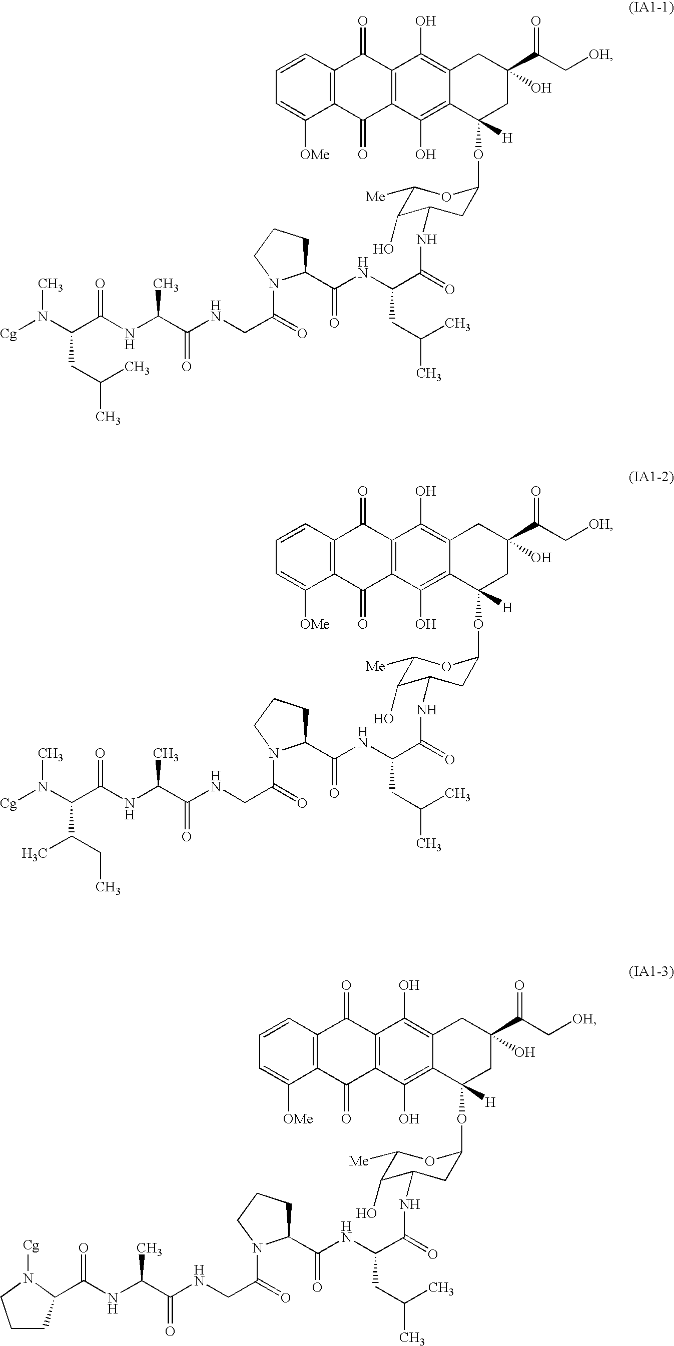 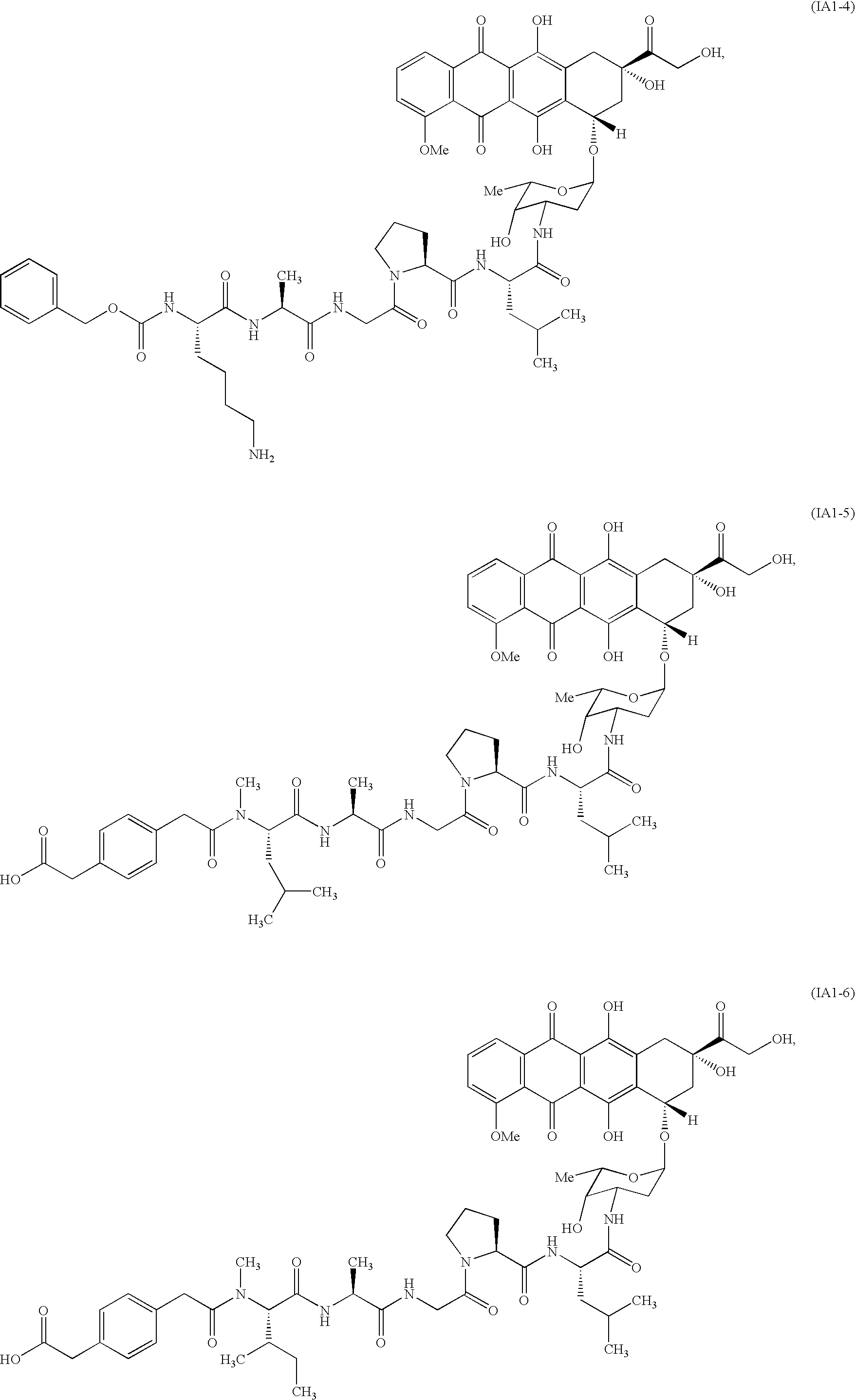 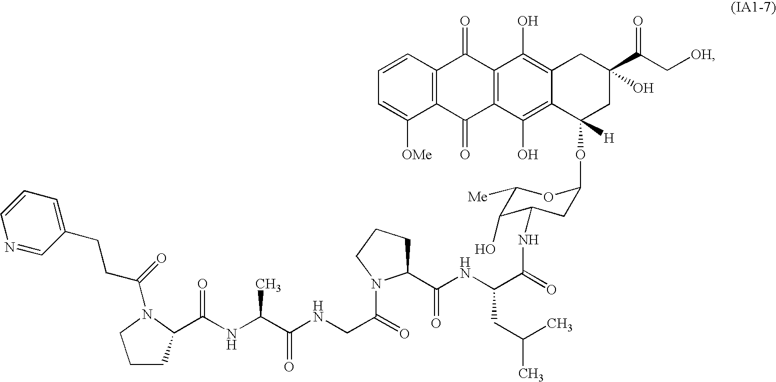
wherein Cg represents a capping group, which blocks degradation of the attached oligopeptide moiety in body fluids.

24. A compound of the formula (I) according to claim 1, which is converted into a conjugate of the formula (IV)
H-Xaa³-Cyt (IV)
wherein Xaa³ and Cyt are as defined in claim 1,
by the catalytic action of human fibroblast activation protein (FAPα), in the immediate environment of a target cell.

25. A method for the treatment of tumors which express FAPα which comprises the administration of a therapeutically effective amount of a compound of the formula (I)
Cg-(Xaa¹)ₘ-Xaa²-Caa-Xaa³-Cyt (I)
or a pharmaceutically acceptable salt thereof, wherein:
Xaa¹ each independently represent any genetically encoded amino acid or the N-alkylated derivative thereof, at least one of which being N-terminally linked to Cg;
Xaa² represents any genetically encoded amino acid or the N-alkylated derivative thereof, which is C-terminally linked to Caa;
Xaa³ represents an amino acid selected from the group consisting of leucine, phenylalanine, isoleucine, alanine, β-alanine, glycine, tyrosine, 2-naphthylalanine and serine, or an N-alkylated derivative thereof, which is C-terminally linked to Cyt;
Caa represents an optionally substituted cyclic amino acid, which is C-terminally linked to Xaa³;
Cg represents a capping group, which blocks degradation of the attached oligopeptide moiety in body fluids;
Cyt represents the residue of a cytotoxic or cytostatic compound; and
m is 0 or an integer from 1 to 6.

26. A pharmaceutical composition comprising a compound of the formula (I)
Cg-(Xaa¹)ₘ-Xaa²-Caa-Xaa³-Cyt (I)
or a pharmaceutically acceptable salt thereof, wherein:
Xaa¹ each independently represent any genetically encoded amino acid or the N-alkylated derivative thereof, at least one of which being N-terminally linked to Cg;
Xaa² represents any genetically encoded amino acid or the N-alkylated derivative thereof, which is C-terminally linked to Caa;
Xaa³ represents an amino acid selected from the group consisting of leucine, phenylalanine, isoleucine, alanine, β-alanine, glycine, tyrosine, 2-naphthylalanine and serine, or an N-alkylated derivative thereof, which is C-terminally linked to Cyt;
Caa represents an optionally substituted cyclic amino acid, which is C-terminally linked to Xaa³;
Cg represents a capping group, which blocks degradation of the attached oligopeptide moiety in body fluids;
Cyt represents the residue of a cytotoxic or cytostatic compound; and
m is 0 or an integer from 1 to 6,
and one or more pharmaceutically acceptable excipients.
